(19) United States
(12) Patent Application Publication
(10) Pub. No.: US 2017/0216622 A1
(43) Pub. Date: Aug. 3, 2017

(54) TREATMENT PLANNING DEVICE, TREATMENT PLANNING METHOD, CONTROL DEVICE, AND PARTICLE BEAM TREATMENT SYSTEM

(71) Applicant: Hitachi, Ltd., Tokyo (JP)

(72) Inventors: Shinichiro FUJITAKA, Tokyo (JP); Shusuke HIRAYAMA, Tokyo (JP); Masumi UMEZAWA, Tokyo (JP)

(21) Appl. No.: 15/418,838

(22) Filed: Jan. 30, 2017

(30) Foreign Application Priority Data
Jan. 28, 2016 (JP) 2016-013903

(51) Int. Cl. A61N 5/10

(52) U.S. Cl. CPC A61N 5/103; A61N 5/1043; A61N 2005/1087

(57) ABSTRACT

A spot determination unit classifies an irradiation region to be irradiated with a charged particle beam into a plurality of layers in an irradiation direction of the charged particle beam, and arranges a plurality of irradiation spots in the plurality of layers. The irradiation spots are classified into groups in accordance with at least either a distance between one irradiation spot and another irradiation spot which are arranged in the same layer or a target irradiation dose of each irradiation spot. A plan is prepared for continuously emitting the charged particle beam while the irradiation position is changed from an irradiation spot belonging to a certain group to a subsequent irradiation spot, and so as to stop emitting the charged particle beam while the irradiation position is changed from an irradiation spot belonging to a certain group to an irradiation spot belonging to another group located in the same layer.

### TECHNICAL FIELD

The present invention relates to a particle beam treatment device which treats a cancer by irradiating an affected area with a charged particle beam accelerated by a synchrotron or cyclotron particle beam accelerator.

### BACKGROUND ART

As a method of irradiating an affected area with a charged particle beam in particle beam treatment, a scanning irradiation method is known in which an irradiation target is irradiated with a charged particle while being directly scanned. In particle beam scanning irradiation, in order to irradiate the affected area with a uniform irradiation dose, a plurality of scanning methods are used. In scanning irradiation method called dispersed spot irradiation, irradiation spots to be irradiated with a beam are arranged in the affected area, and a treatment planning device determines a target irradiation dose for each irradiation spot. When the affected area is irradiated, an irradiation position and an irradiation dose of the charged particle beam are measured, and the determined irradiation spots are irradiated with the beam as much as a predetermined irradiation dose. If one irradiation spot is completely irradiated with the beam, a beam is stopped (turned off) once, and is moved to the subsequent irradiation spot. The beam is turned on again so as to irradiate the subsequent irradiation spot. This procedure is repeatedly performed on all of the irradiation spots, thereby completing the irradiation. In order to change energy in a depth direction of the affected area, energy of the charged particle beam is changed by an accelerator, thereby changing the irradiation spot in the depth direction.

In addition, as another scanning method, an irradiation method called continuous beam irradiation is known. This method is the same as the dispersed spot irradiation in that the beam moves to the subsequent irradiation spot if the irradiation spot is completely irradiated as much as the irradiation dose. However, according to the irradiation method, even when beam moves between the irradiation spots, the beam moves while irradiating the irradiation spot with the beam. In order to perform scanning irradiation using the continuous beam, it is necessary not only to consider the irradiation dose for irradiating a portion between the irradiation spots with the charged particle beam, but also to cause a treatment planning device to calculate an irradiation plan which enables the affected area to be irradiated with a uniform irradiation dose. PTL 1 discloses the following method. In a case where the continuous beam irradiation is performed, when the irradiation spots are separated from each other, the irradiation spots are classified into groups, thereby determining a scanning route.

### CITATION LIST

### Patent Literature

PTL 1: Japanese Patent No. 5791793

### SUMMARY OF INVENTION

### Technical Problem

According to a technique disclosed in PTL 1, when the irradiation spots are separated from each other, the irradiation has to be performed by lowering beam intensity so that the irradiation dose for the portion between the irradiation spots does not increase. In addition, in a case where the irradiation spots which have mutually different magnitudes of the target irradiation dose are mixed with each other, it is necessary to perform the irradiation in accordance with the irradiation spot whose target irradiation dose is small. For these reasons, in a case where the continuous beam irradiation is performed, the irradiation cannot be performed by raising the beam intensity. Consequently, a treatment time tends to be lengthened.

### Solution to Problem

In order to solve the above-described problem, the present invention adopts configurations described in Claims, for example.

The present application includes a plurality of means for solving the above-described problem. As an example, there is provided a treatment planning device including a spot determination unit that classifies an irradiation region to be irradiated with a charged particle beam into a plurality of layers in an irradiation direction of the charged particle beam, and that arranges a plurality of irradiation spots which serve as irradiation positions of the charged particle beam, in the layers, a group classification unit that classifies the irradiation spots into groups in accordance with at least either a distance between one irradiation spot and another irradiation spot which are arranged in the same layer or a target irradiation dose of each irradiation spot, and a planning unit that prepares a plan so as to continuously emit the charged particle beam while the irradiation position is changed from an irradiation spot to a subsequent irradiation spot belonging to a certain group, and so as to stop emitting the charged particle beam while the irradiation position is changed from an irradiation spot belonging to a certain group to another irradiation spot belonging to another group located in the same layer as that of the certain group.

### Advantageous Effects of Invention

According to the present invention, a treatment time can be shortened in particle beam scanning irradiation.

### BRIEF DESCRIPTION OF DRAWINGS

FIG. 1 is a view illustrating an overall configuration of a particle beam treatment system.

FIG. 2 is a view illustrating a particle beam scanning irradiation nozzle.

FIG. 3 is a view illustrating a layer irradiated with the same energy, a charged particle beam, and irradiation spots, when scanning irradiation is performed on an affected area.

FIG. 4 is a view illustrating irradiation dose distribution in a depth direction when the scanning irradiation is performed on the affected area.

FIG. 5 is a view illustrating continuous beam irradiation.

FIG. 6 is a view illustrating dispersed spot irradiation.

FIG. 7 is a view illustrating control for the continuous beam irradiation.

FIG. 8 is a view illustrating control for the dispersed spot irradiation.

FIG. 9 is a flowchart illustrating a first embodiment according to the present invention.

FIG. 10 is a view illustrating an arrangement of the irradiation spots.

FIG. 11 is a view illustrating group classification in accordance with a distance relationship between the irradiation spots.

FIG. 12 is a view illustrating scanning irradiation in which continuous beam irradiation and dispersed spot irradiation are combined with each other according to a position embodiment of the present invention.

FIG. 13 is a view for describing moving between the irradiation spots and stopping at the irradiation spot when the continuous beam irradiation is performed.

FIG. 14 is a view illustrating continuous beam irradiation in the related art.

FIG. 15 is a flowchart illustrating the first embodiment according to the present invention.

FIG. 16 is a flowchart illustrating a second embodiment according to the present invention.

FIG. 17 is a flowchart illustrating a third embodiment according to the present invention.

### DESCRIPTION OF EMBODIMENTS

Specific embodiments according to the present invention will be described in detail with reference to the drawings.

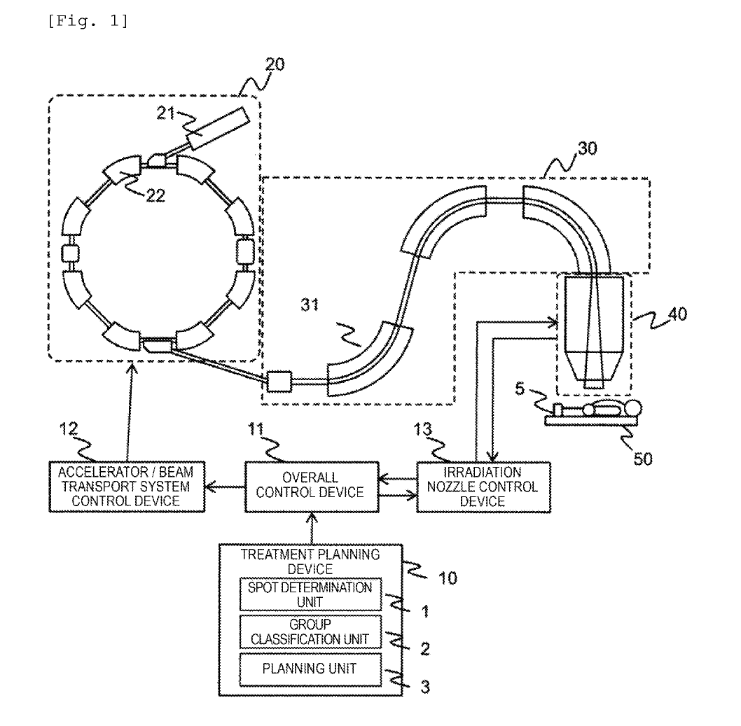

FIG. 1 illustrates an overall configuration of a particle beam treatment system according to an embodiment of the present invention. The particle beam treatment system includes an accelerator 20 that accelerates a charged particle beam (hereinafter, referred to as a beam) 90, a beam transport system 30 that transports the accelerated beam 90 to an irradiation nozzle, an irradiation nozzle 40 that irradiates an affected area with the beam, a treatment table 50, a treatment planning device 10 that prepares a treatment plan, an overall control device 11, an accelerator/beam transport system control device 12, and an irradiation nozzle control device 13. Here, the treatment planning device 10 includes a spot determination unit 1, a group classification unit 2, and a planning unit 3. In addition, the accelerator 20 includes a beam emitter 21 and a synchrotron accelerator 22. The beam 90 accelerated up to 60% to 70% of light speed by the accelerator 20 is transported to the irradiation nozzle 40 while being deflected in vacuum in a magnetic field by a deflection electromagnet 31 disposed in the beam transport system 30. The beam 90 is shaped in the irradiation nozzle 40 so as to match a shape of an irradiation region, and an irradiation target is irradiated with the beam 90. For example, the irradiation target is an affected area 51 of a patient 5 lying on the treatment table 50.

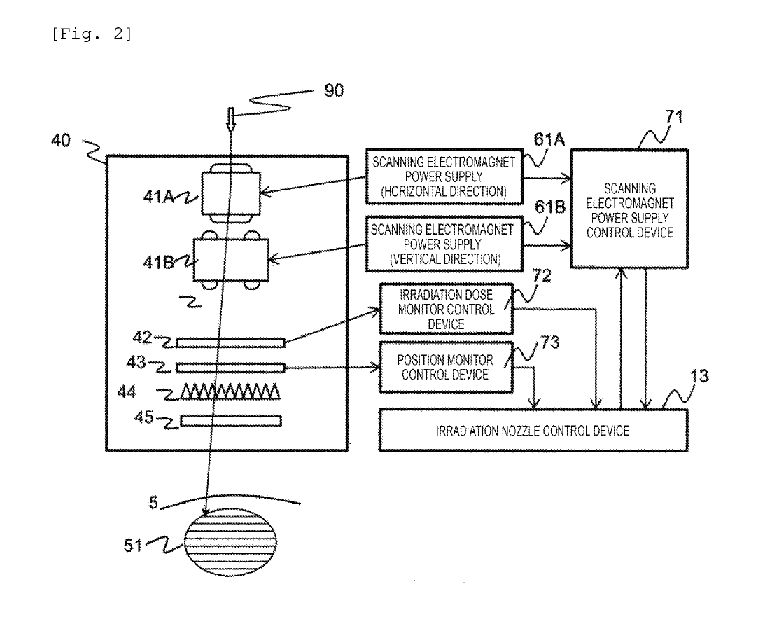

FIG. 2 illustrates the irradiation nozzle 40 for particle beam scanning according to an embodiment of the present invention. In the irradiation nozzle 40, scanning electromagnets 41A and 41B for horizontal and vertical directions scan the inside of a two-dimensional plane with the beam 90. The affected area 51 is irradiated with the beam 90 used in the scanning performed by the scanning electromagnets 41A and 41B. An irradiation dose monitor 42 measures an irradiation dose of the beam 90 used in irradiating each irradiation spot. An irradiation dose monitor control device controls the irradiation dose for irradiating each irradiation spot. A position monitor 43 measures a beam position (for example, a position of the center of gravity) of each irradiation spot. A position monitor control device 73 calculates a position and a width of the irradiation spot, based on data of the beam position measured by the position monitor 43, and confirms an irradiation position of the beam 90. When necessary, a ridge filter 44 is used in order to thicken a Bragg peak. In addition, an arrival position of the beam 90 may be adjusted by inserting a range shifter 45.

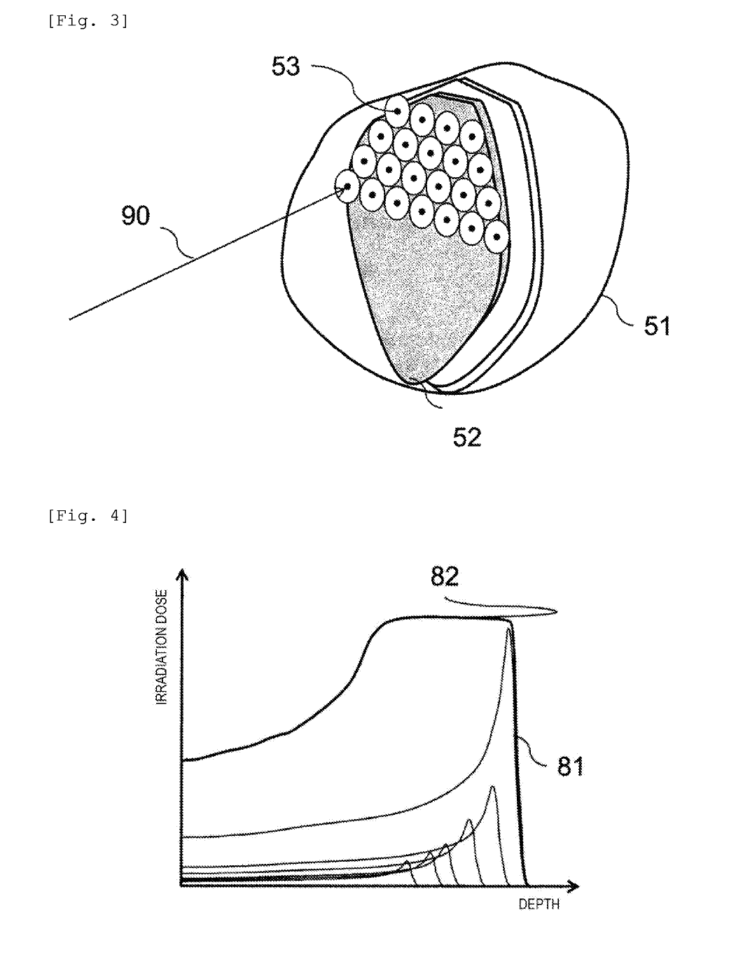

When the scanning irradiation is performed, the treatment planning device 10 illustrated in FIG. 1 calculates positions of the irradiation spots and a target irradiation dose for each irradiation spot in advance in order to irradiate the affected area with a uniform irradiation dose. FIG. 3 illustrates particle beam scanning irradiation. The affected area 51 is classified into layers 52, and the inside of each layer 52 is irradiated with the beam 90 having the same energy. Irradiation spots 53 are arranged inside the layer 52.

Data for each patient which is calculated by the treatment planning device 10 illustrated in FIG. 1 is sent to the overall control device 11 of the particle beam treatment system illustrated in FIG. 1. An energy changing signal, a beam emission start signal, or a beam emission stop signal is output from the overall control device 11 to the accelerator/beam transport system control device 12. A coordinate value and an irradiation dose of each irradiation spot are sent from the overall control device 11 to the irradiation nozzle control device 13. The coordinate value of the irradiation spot is converted into an excitation current value of the scanning electromagnets 41A and 41B. The excitation current value is sent to a scanning electromagnet power supply control device 71 illustrated in FIG. 2.

If a certain irradiation spot 53 arranged by the treatment planning device is irradiated with the beam 90 of a determined irradiation dose, a subsequent irradiation spot is irradiated. If a certain layer 52 is completely irradiated, the subsequent layer 52 is irradiated. First, beam energy is changed in order to change the irradiation position in a beam irradiation direction, that is, in a depth direction of the affected area. If the beam energy is changed, a position of the beam arriving at the inside a body is changed. The charged particle beam having high energy arrives at a deep position inside the body, and the charged particle beam having low energy arrives at only a shallow position inside the body. When the particle beam scanning irradiation is performed, the beam energy is changed in order to form uniform irradiation dose distribution in the depth direction, and the irradiation dose is properly distributed, thereby forming a spread out Bragg peak (SOBP) in the depth direction. Each energy irradiation dose is properly distributed. In this manner, energy Bragg curves 81 are superimposed together, thereby forming uniform irradiation dose distribution SOBP82 in the depth direction as illustrated in FIG. 4.

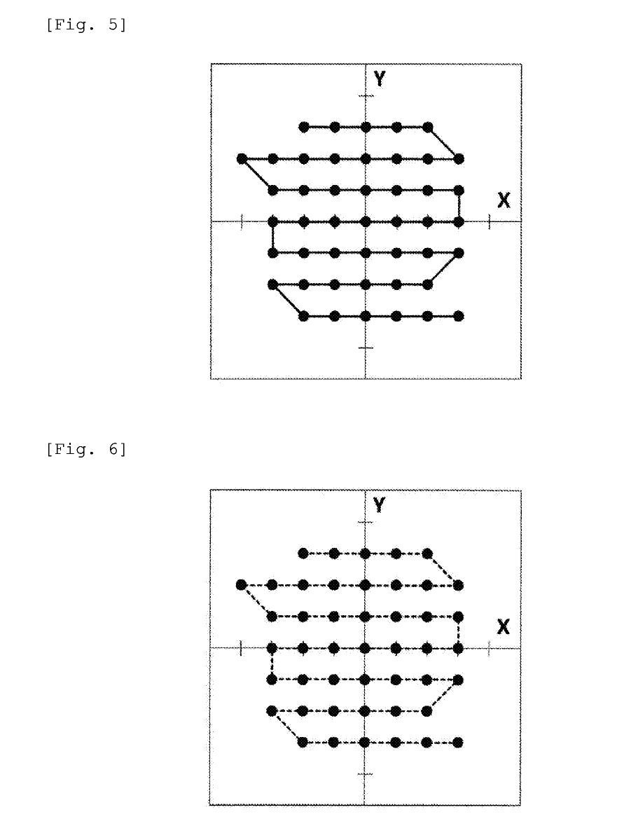

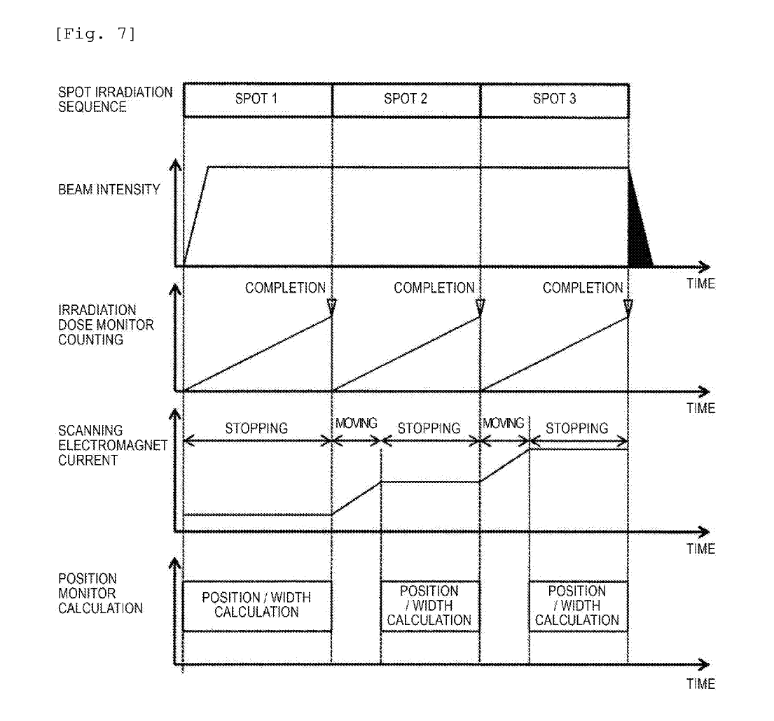

Next, irradiation in the horizontal direction for the scanning irradiation will be described. In the treatment planning device 10, the irradiation spots for irradiating the affected area with a uniform irradiation dose are arranged for each energy of the beam as illustrated in FIG. 3. FIG. 5 illustrates the scanning irradiation using continuous beam irradiation. FIG. 6 illustrates the scanning irradiation using dispersed spot irradiation. A black point represents the irradiation spot. A solid line represents that a beam moves between the irradiation spots while being turned on. A dotted line represents that the beam moves between the irradiation spots while being turned off. As illustrated in FIG. 5, in a case of the continuous beam irradiation, if an irradiation spot is irradiated with the beam as much as the irradiation dose of each irradiation spot which is determined by a treatment plan, the beam moves to the subsequent irradiation spot without turning off the beam. Therefore, the irradiation dose for the irradiation spots is a sum of the irradiation dose used in the irradiation while the beam moves between the irradiation spots and the irradiation dose used in the irradiation while the beam stops at the irradiation spots. The treatment plan corresponding to the continuous beam irradiation determines in advance a scanning route for scanning the irradiation spots as illustrated by the solid line in FIG. 5. In a case of the dispersed spot irradiation illustrated in FIG. 6, the same scanning route as that of the continuous beam irradiation in FIG. 5 is illustrated. However, the beam moves to the subsequent irradiation spot while being turned off when the beam moves between the irradiation spots. Therefore, the irradiation dose is provided for only the spots illustrated by the black point in FIG. 6.

In the continuous beam irradiation, the beam moves between the irradiation spots while the beam is also turned on between the irradiation spots. Accordingly, it is necessary to prepare an irradiation plan for irradiating the affected area with a uniform irradiation dose also in view of the irradiation dose given by the irradiation while the beam moves between the spots. For example, a method may be employed which optimizes the irradiation dose by arranging virtual irradiation spots between the irradiation spots and causing the virtual spots to represent the irradiation dose during the movement. In addition, in the continuous beam irradiation, in order to consider the irradiation dose given by the beam in the irradiation while the beam moves between the irradiation spots, it is necessary to determine a scanning route for scanning the irradiation spots inside a layer to be irradiated with the same energy. For example, a method may be employed which determines the scanning route so as to minimize the scanning distance for scanning the irradiation spots by using a traveling salesman algorithm.

Controls for the continuous beam irradiation and the dispersed spot irradiation will be described in detail. FIG. 7 illustrates a time chart of the continuous beam irradiation. As an example, FIG. 7 illustrates irradiation at three spots from a spot 1 to a spot 3. The accelerator/beam transport system control device 12 illustrated in FIG. 1 instructs the accelerator 20 to irradiate the spots with predetermined beam intensity. If the beam irradiation starts, an ionization output of the irradiation dose monitor 42 inside the irradiation nozzle 40 is converted into pulses by the irradiation dose monitor control device 72, and a pulse count value starts to increase. If a predetermined irradiation dose is achieved, the irradiation dose monitor control device 72 sends a completion signal to the irradiation nozzle control device 13, and the irradiation of the spot is completed. In accordance with the completion signal of the irradiation dose monitor control device 72, the irradiation nozzle control device 13 sends to the scanning electromagnet power supply control device 71 a signal to move to the subsequent spot, and the irradiation nozzle 40 starts to move to the subsequent spot. If a current value of the subsequent spot is attained, the scanning electromagnet power supply control device 71 sends a movement completion signal to the irradiation nozzle control device 13. After receiving a stop completion signal, the position monitor control device 73 obtains an output from the position monitor 43, and starts to calculate a position and a width of the beam. In accordance with an irradiation dose completion signal, the position monitor control device 73 completes the calculation of the position and the width of the beam. The irradiation nozzle control device 13 determines whether or not a predetermined position is irradiated. As a result of the determination, when the position and the width of the beam are greatly deviated, the beam irradiation is stopped. Hitherto, a flow in the control for the continuous beam irradiation has been described.

At the final spot of the continuous beam irradiation, the accelerator 20 has a limited response time. Thus, in some cases, a delayed charge may be generated after the emission stop signal of the beam is output. Predictive control is performed on the delayed charge, and the beam is turned off beforehand. In this manner, it is possible to control the continuous beam irradiation so as to provide the predetermined irradiation dose.

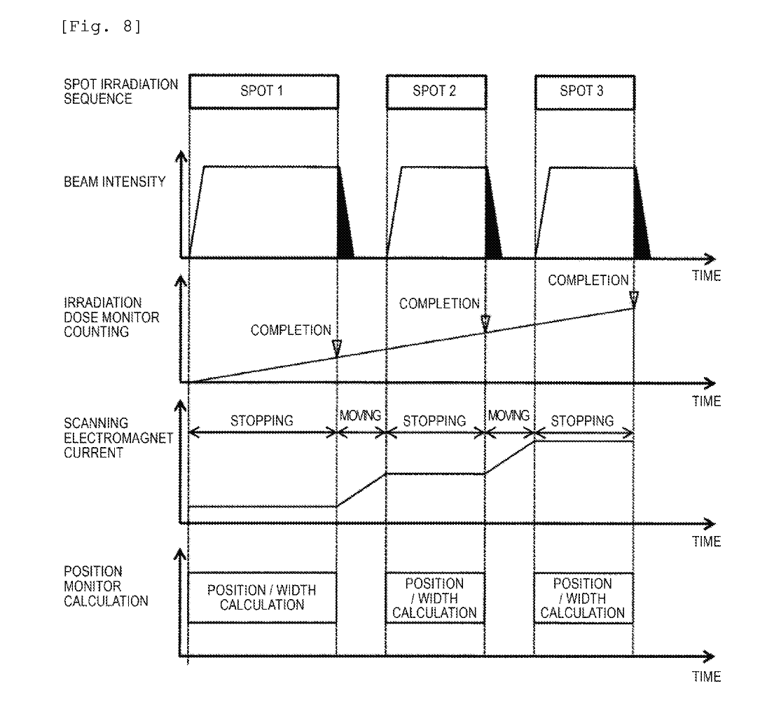

Next, FIG. 8 illustrates a time chart of the dispersed spot irradiation. The dispersed spot irradiation is the same as the continuous beam irradiation in that the beam moves to the subsequent spot in response to the irradiation dose completion signal by changing the current of the scanning electromagnet. In the dispersed spot irradiation, after receiving the irradiation dose completion signal from the irradiation dose monitor control device 72, the irradiation nozzle control device 13 sends an instruction to turn off the beam to the accelerator/beam transport system control device 12 via the overall control device 11, thereby turning off the beam. Thereafter, after the turn-off of the beam is completed, the accelerator/beam transport system control device 12 sends a movement start signal to the irradiation nozzle control device 13 via the overall control device 11. The irradiation nozzle control device 13 receives the movement start signal, and sends a signal to move to the subsequent spot to the scanning electromagnet power supply control device 71. After receiving a movement completion signal of the scanning electromagnet power supplies 61A and 61B, the irradiation nozzle control device 13 sends an instruction to turn on the beam to the accelerator/beam transport system control device 12. In this manner, the beam irradiation starts again, and the subsequent spot starts to be irradiated. The calculation of the position and the width of the beam starts after the movement completion signal is received. The calculation is completed after the completion signal of the irradiation spot is received. Since the beam is turned off between the respective spots, a delay component is present in the irradiation dose compared to the response time of the accelerator 20. This causes the irradiation dose at each spot to increase as much as the amount of the delay component. Therefore, in the dispersed spot irradiation, all of the irradiation doses are integrated and managed, thereby ensuring accuracy in the irradiation dose.

Hitherto, the controls for the continuous beam irradiation and the dispersed spot irradiation have been described. In the continuous beam irradiation, even while the beam moves between the irradiation spots, the beam irradiation is performed. In contrast, in the dispersed spot irradiation, while the beam moves between the irradiation spots, the beam irradiation is not performed. Accordingly, the continuous beam irradiation can shorten a treatment time compared to the dispersed spot irradiation. On the other hand, since the dispersed spot irradiation does not have the irradiation dose for irradiating a portion between the spots, each spot can be very accurately irradiated.

If irradiation can be performed in both the continuous beam irradiation and the dispersed spot irradiation according to the above-described configurations, it is desirable to perform both of these from a viewpoint of the treatment time and accurate irradiation. Since the control device corresponding to two irradiation methods of the dispersed spot irradiation and the continuous beam irradiation is provided, more flexible scanning irradiation can be realized. Here, the same irradiation nozzle may be configured so that both the dispersed spot irradiation and the continuous beam irradiation can be performed. Since the irradiations can be performed using the same irradiation nozzle, it is possible to shorten a time needed to switch between the dispersed spot irradiation and the continuous beam irradiation.

### Embodiment 1

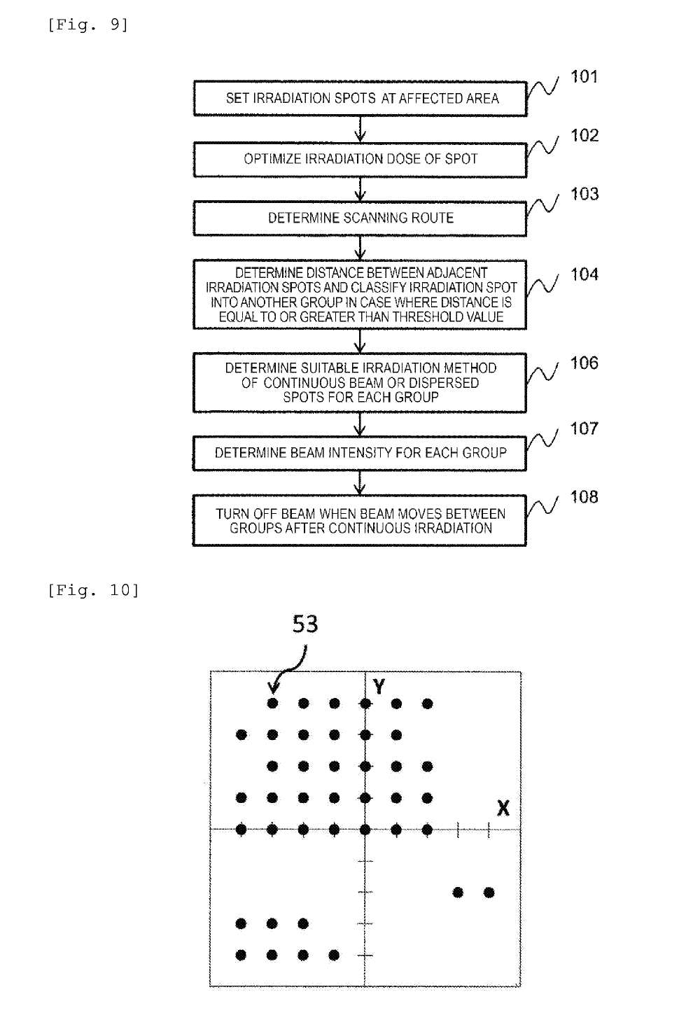

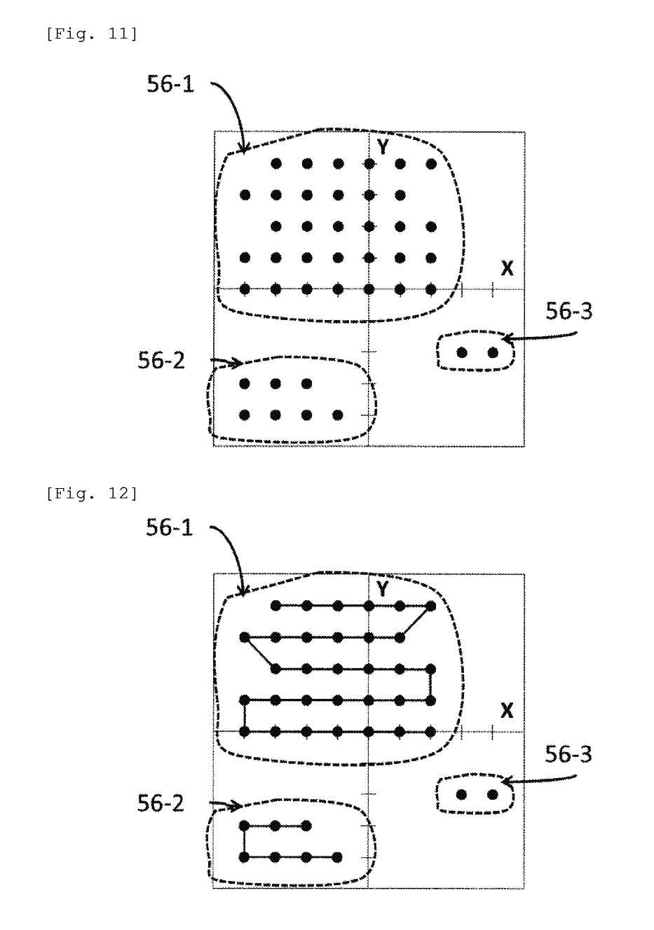

A first embodiment according to the present invention will be described with reference to the drawings. FIG. 9 illustrates a flowchart according to the present embodiment. When the treatment planning device calculates an irradiation plan for the continuous beam irradiation, the spot determination unit 1 included in the treatment planning device first classifies the affected area 51 into the layers 52, and sets the irradiation spots 53 as illustrated in FIG. 10 (Step 101). Next, through repeated calculation using the quasi-Newton method, the irradiation dose is optimized so that the irradiation dose for the affected area reached a predetermined irradiation dose (Step 102). As a result, a target irradiation dose for each irradiation spot is determined. Next, a scanning route inside the layer is determined (Step 103). The traveling salesman algorithm or the like is used in determining the scanning route, thereby reducing the total scanning distance. After the scanning route is determined, the group classification unit 2 calculates a distance between the adjacent spots, and performs processing on the spots which are separated from each other with a certain distance or farther so as to be classified into another group (Step 104). Through the processes in Steps 103 and 104, the irradiation spots inside the layer 52 are classified into a plurality of groups. Here, a group includes one or more irradiation spots. For example, group classification is performed as illustrated in FIG. 11.

If the irradiation spots inside the layer 52 are classified into the plurality of groups, the planning unit 3 determines whether to perform the continuous beam irradiation or the dispersed spot irradiation on each group (Step 106). In groups 56-1 and 56-2 in FIG. 11, many spots are densely present. Accordingly, the continuous beam irradiation is performed. In a group 56-3 in FIG. 11, only two spots are present. Accordingly, the dispersed spot irradiation is performed. In the continuous beam irradiation, the irradiation position is changed in a state where the beam is continuously emitted. Therefore, compared to a case where the dispersed spot irradiation is performed on all of the irradiation spots, it is possible to shorten the treatment time by selecting a proper irradiation method.

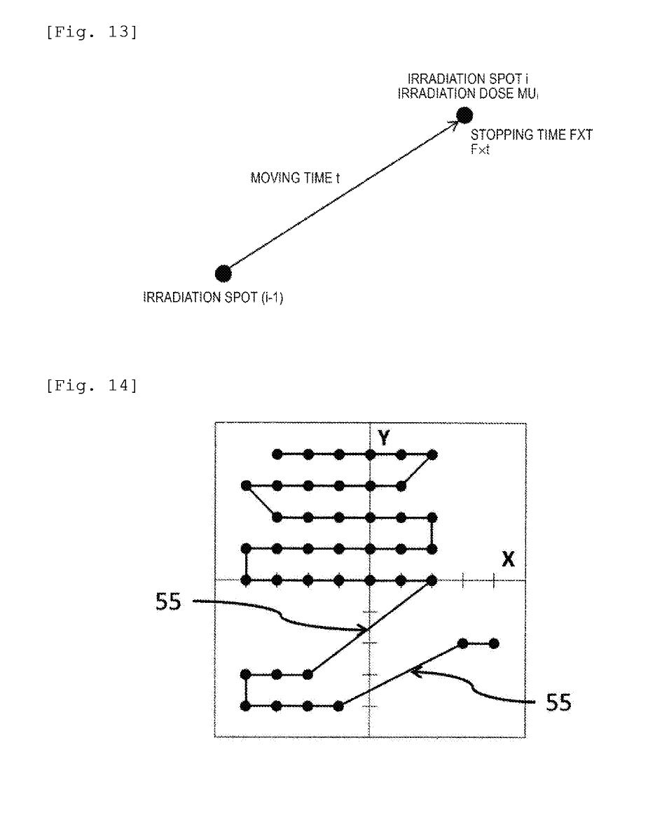

If the irradiation method is determined as illustrated in FIG. 12, with regard to the group on which the continuous beam irradiation is performed, beam intensity is determined for each group (Step 107). In this case, the beam intensity may be changed for each group.

In accordance with the above-described flow illustrated in FIG. 9, the treatment planning device 10 determines the scanning route and the irradiation method for irradiating the irradiation spots inside the layer. When necessary, in view of the irradiation dose during the movement, the irradiation dose is optimized again.

A method of determining the beam intensity in the continuous beam irradiation in Step 107 will be described. In the continuous beam irradiation, even while the beam moves between the irradiation spots, the beam irradiation is continuously performed. Accordingly, it is necessary to manage the irradiation dose while the beam moves between the irradiation spots. In the continuous beam irradiation, a moving time between the irradiation spots is determined by the distances and the scanning speed between the irradiation spots. Accordingly, the irradiation dose for irradiating the portion between the irradiation spots is managed by controlling the beam intensity of the beam emitted from the accelerator 20 so as to be constant. The distance between the irradiation spots is determined by the treatment plan, based on a shape of the affected area. In addition, the scanning speed is determined if the beam energy is determined by the design of the scanning electromagnets 41A and 41B arranged in the irradiation nozzle 40.

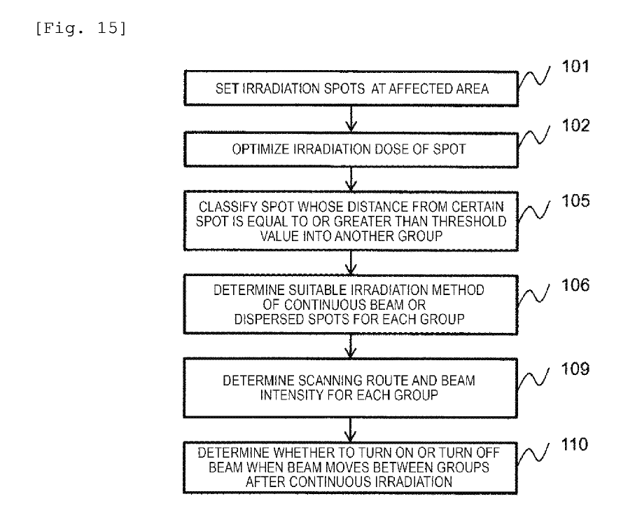

FIG. 13 illustrates two adjacent irradiation spots, and the solid line represents the scanning route through which the beam moves while being turned on. In the continuous beam irradiation, in view of a ratio of the movement of the beam between the irradiation spots and stopping at the irradiation spots, the beam intensity for irradiating each layer is determined. First, the moving time of the beam between the irradiation spots can be calculated in such a way that the interval between the irradiation spots is divided by the scanning speed. The scanning speed of the irradiation nozzle varies between the X-direction and the Y-direction. If the scanning speeds in the X-direction and the Y-direction are respectively set to Vx and Vy and the spot intervals in the X-direction and the Y-direction are respectively set to Lx and Ly, moving times tx and ty in the X-direction and the Y-direction are respectively calculated as follows.

$$\begin{matrix}[ Expression 1 ] & \\ {t}_{X} = \frac{{L}_{X}}{{V}_{X}} {t}_{Y} = \frac{{L}_{Y}}{{V}_{Y}} & ( 1 )\end{matrix}$$

A moving time t between the adjacent irradiation spots illustrated in FIG. 12 is maximized in the moving times in the X-direction and the Y-direction, and is expressed as follows.

[Expression 2]

t=max(t_{X},t_{Y}) (2)

Here, if the ratio between the stopping and the movement is set to F (value obtained in such a way that the stopping time is divided by the moving time) and the irradiation dose for the subsequent i-th number of irradiation spot is set to MUi, the beam has constant intensity, and the irradiation spot is irradiated with the irradiation dose MUi during a period of the moving time t and the stopping time Ft. Accordingly, beam intensity Ii of the beam for irradiating the i-th number of irradiation spot is obtained as follows.

$$\begin{matrix}[ Expression 3 ] & \\ {I}_{i} = \frac{{MU}_{i}}{( 1 + F ) t} & ( 3 )\end{matrix}$$

This is repeatedly calculated for all of the irradiation spots on a layer irradiated with the same energy so as to select the minimum current. In this manner, beam intensity I for the layer is determined.

$$\begin{matrix}[ Expression 4 ] & \\ I = {min}_{i \text{:} all irradiatuion spots inside layer} ( {I}_{j} ) & ( 4 )\end{matrix}$$

In this manner, all of the irradiation spots inside the layer can be irradiated at the ratio F of the movement and stopping or greater. For example, F (stopping time/moving time)=3 is selected and calculated. In this manner, for all of the irradiation spots inside the layer, it is possible to secure the stopping time which is three times or longer than the moving time. The beam emitted from the accelerator 20 is controlled so as to have constant beam intensity by controlling the accelerator 20. However, if the beam intensity is observed in detail in terms of time, the beam intensity fluctuates at all times with a certain fluctuation width. In the present embodiment, the stopping time is provided with a margin through the above-described algorithm. Therefore, even in a case where the current intensity of the beam increases, it is possible to prevent the irradiation dose from being completely consumed during the moving time.

However, if F increases, a possibility that the irradiation dose may be completely consumed during the moving time decreases, and stable irradiation can be performed in the continuous beam irradiation. In contrast, the beam intensity is lowered, based on Expression (3). That is, beam intensity I for irradiating the layer is also lowered. Since a treatment time T is a value obtained in such a way that all of the irradiation doses are divided by the beam intensity, there is a problem in that the treatment time T is lengthened.

$$\begin{matrix}[ Expression 5 ] & \\ T = \frac{{\sum }_{i \text{:} all irradiation spots} {MU}_{i}}{J} & ( 5 )\end{matrix}$$

In particular, in the continuous beam irradiation, in a case where scanning is performed while the beam is turned on between remotely separated irradiation spots, the moving time t is lengthened in Expressions (1) and (2). Therefore, it is necessary to lower the beam intensity, based on Expression (3).

In FIG. 14, two remotely separated irradiation spots 55 are present. As illustrated in FIG. 13, in a case where the beam moves between the spots while the continuous beam irradiation is performed, it is necessary to lower the beam intensity. Therefore, in Step 108, the planning unit 3 always turns off the beam in a case where the beam moves between the groups after the continuous beam irradiation is performed. In this manner, even if there is a remotely separated spot in a case of the continuous beam irradiation, the continuous beam irradiation can be performed without lowering the beam intensity, and the treatment time can be shortened.

According to the present embodiment, the irradiation spot whose distance from a certain irradiation spot is equal to or greater than a threshold value is classified into another group, and the beam emission is stopped between the groups. Accordingly, even in a case where an important organ is present between the remotely separated irradiation spots, the beam is turned off on the route passing through the important organ. The position of the important organ does not limit how to select the initial irradiation spot (starting point) and the last irradiation spot (end point) inside each group. Therefore, a shorter scanning route inside each group can be selected, and the beam scanning time can be shortened. Accordingly, the treatment time can be shortened.

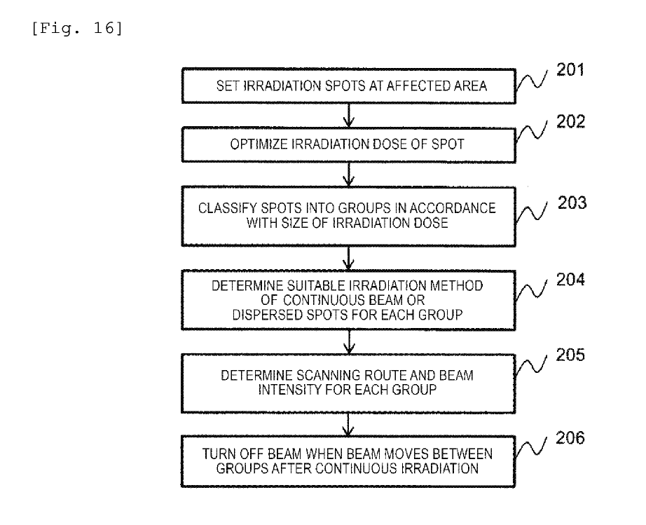

According to the present embodiment, as illustrated in FIG. 9, the scanning route which indicates the beam irradiation order is determined (Step 103). Thereafter, a distance between the adjacent irradiation spots is determined. In a case where the distance is equal to or greater than a threshold value, the irradiation spot is classified into another group, thereby grouping the irradiation spots (Step 104). However, instead of Step 103 and Step 104, as illustrated in FIG. 15, in view of a distance relationship between a plurality of irradiation spots inside the layer, a group classification process (Step 105) may be performed on the irradiation spots. Specifically, the group classification based on the distance relationship means that the irradiation spots inside the layer are classified into the groups by repeating the following process. If the distance between two selected irradiation spots is smaller than the threshold value, the irradiation spots are classified into the same group. If the distance is equal to or greater than the threshold value, the irradiation spots are classified into another group. The irradiation method is selected similarly to Step 106 in FIG. 9. Next, the scanning route and the beam intensity for each group are determined (Step 109), and the starting point and the endpoint for each group are also determined. In this case, the beam intensity for each group may be changed. Therefore, the distance from the end point of the group before the movement to the starting point of the group after then movement is used so as to determine whether to turn on or off the beam during the movement between the groups after the continuous beam irradiation is performed (Step 110). In a case of FIG. 10, the scanning route for each group can be determined. Therefore, the beam scanning time can be further shortened, and the treatment time can be shortened.

### Embodiment 2

According to a second embodiment of the present invention, the irradiation spots are classified into groups depending on a target irradiation dose.

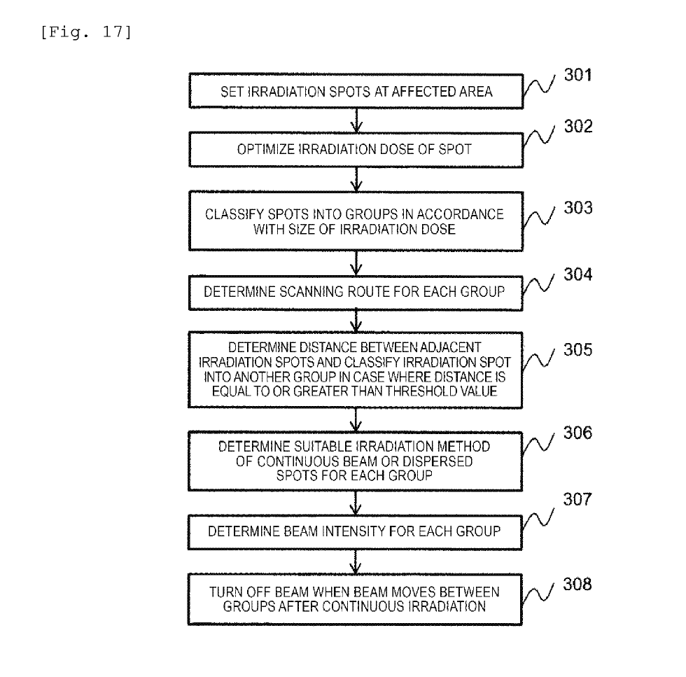

FIG. 16 illustrates a flowchart according to the second embodiment of the present invention. Similarly to Embodiment 1, the spot determination unit 1 included in the treatment planning device 10 first sets the irradiation spot 53 (Step 201). Thereafter, the irradiation dose is optimized (Step 202), and the target irradiation dose for each spot is determined. These processes correspond to Step 101 and Step 102 in Embodiment 1. Next, the group classification unit 2 classifies the spots into the groups in accordance with a magnitude of the target irradiation dose for each spot (Step 203). For example, in a case where the spots are classified into two groups, a spot having the more target irradiation dose than a certain target irradiation dose is classified into a first group, and a spot having the less target irradiation dose than the certain target irradiation dose is classified into a second group. The number of groups is not limited to two. The spots may be classified into two or more groups.

Next, based on the number of the irradiation spots included in each group, it is determined whether to perform the continuous beam irradiation or the dispersed spot irradiation (Step 204). With regard to the group on which the continuous beam irradiation is performed in Step 204, the scanning route and the beam intensity are determined (Step 205) similarly to Embodiment 1. The beam intensity is determined as described in Embodiment 1. Step 204 and Step 205 respectively correspond to Step 106 and Step 109 in Embodiment 1. In Step 206, in a case where the continuous beam irradiation is performed on irradiation spots of a certain group, the planning unit 3 causes the beam to move after turning off the beam when the irradiation of the last spot is completed.

As described above, in the second embodiment, the irradiation spots are classified into the groups, based on the magnitude of the target irradiation dose. As a result, in the group having the more target irradiation dose to be given in the continuous beam irradiation, MUi in Expression (3) increases, thereby enabling the beam intensity to be further raised compared to the related art. In the group having the less target irradiation dose, the beam intensity does not vary compared to the related art. As a result, the beam intensity of the continuous beam irradiation can be further raised compared to the related art. Therefore, the treatment time can be shortened.

In addition, in the scanning irradiation, a uniform irradiation dose distribution is formed by superimposing multiple irradiation dose distributions using thin beams on each other. Accordingly, when the affected area is in respiratory movement, it is an effective way to perform repaint irradiation for irradiating the same spot with the beam multiple times. However, when the repaint irradiation is performed using the continuous beam, the spot having the more irradiation dose and the spot having the less irradiation dose are mixed with each other. Accordingly, if the repaint irradiation is performed using the same beam intensity, the stopping time is shortened at the spot having the less target irradiation dose. If the stopping time cannot be secured, it is the only method to secure the stopping time by lowering the beam intensity. However, if the beam intensity is lowered, the treatment time is lengthened.

According to the present embodiment, the spot having the less target irradiation dose is classified into the dispersed spot, and is separated from the spot on which the continuous beam irradiation is performed. In this manner, it is no longer necessary to lower the beam intensity even when the repaint irradiation is performed using the continuous beam. In this way, the group classification is performed in accordance with the target irradiation dose, thereby selecting a suitable irradiation method of the continuous beam or the dispersed spot. Accordingly, without prolonging the treatment time even when the repaint irradiation is performed, it is possible to form a satisfactory irradiation dose distribution.

In addition, the dispersed spot irradiation or the continuous beam irradiation is selected for each group. In this manner, the repaint irradiation can be more freely performed. Therefore, the irradiation dose distribution can be satisfactorily formed for a moving target, and the treatment time can be further shortened compared to the related art.

### Embodiment 3

According to a third embodiment of the present invention, the irradiation spots are classified into the groups in view of both the distance relationship and the magnitude of the target irradiation dose.

FIG. 17 illustrates a flowchart according to the present embodiment.

Processes until the treatment planning device sets the irradiation spots 53 (Step 301) and the irradiation dose is optimized (Step 302) are the same as those according to Embodiment 1. The processes respectively correspond to Step 101 and Step 102 in Embodiment 1. Next, the group classification unit 2 classifies the spots into the groups in accordance with the magnitude of the target irradiation dose for each spot (Step 303), determines the scanning route for each group (Step 304), calculates the distance between the adjacent irradiation spots, and performs a process for further classifying the irradiation spot separated with a certain distance or farther into another group (Step 305).

If the irradiation spots inside a layer are classified into the plurality of groups, it is determined whether to perform the continuous beam irradiation or the dispersed spot irradiation for each group (Step 306). The beam intensity for each group is determined (Step 307). Similarly to Embodiment 1, the beam is always turned off when the beam moves between the groups after the continuous beam irradiation is performed. The processes respectively correspond to Step 106, Step 107, and Step 108 in Embodiment 1. In Step 307, the beam intensity for each group may be changed.

According to the present embodiment, in the group having the more target irradiation dose to be used for the continuous beam irradiation, the beam intensity can be raised compared to the related art. In addition, even in a case where the important organ is present between the irradiation spots separated from each other with a certain distance or farther inside the group classified in accordance with the target irradiation dose, the beam is turned off on the route passing through the important organ. Accordingly, the position of the important organ does not limit how to select the initial irradiation spot (starting point) and the last irradiation spot (end point) inside each group. Therefore, a shorter scanning route inside each group can be selected, and the beam scanning time can be shortened. Accordingly, the treatment time can be shortened.

In addition, an irradiation method suitable for each group is selected in Step 306. Therefore, the treatment time can be shortened compared to a case where the dispersed spot irradiation is performed on all of the irradiation spots.

In the present embodiment, after the scanning route for each group is determined (Step 304), the distance between the adjacent irradiation spots is calculated, and the process for classifying the irradiation spots separated with a certain distance or farther into another group (Step 305) is performed. However, instead of Step 304 and Step 305, the process for classifying the plurality of irradiation spots inside the layer into the groups may be performed, based on the distance relationship. Specifically, the group classification based on the distance relationship means that the irradiation spots inside the layer are classified into the groups by repeating the following process. If the distance between two selected irradiation spots is smaller than the threshold value, the irradiation spots are classified into the same group, and if the distance is equal to or greater than the threshold value, the irradiation spots are classified into another group.

In addition, in the present embodiment, after the irradiation spots are classified into the groups in accordance with the magnitude of the target irradiation dose, the irradiation spots are classified into the groups in accordance with the distance between the irradiation spots. However, the process order may be reversely performed.

In addition to the synchrotron accelerator 22 described in Embodiments 1 to 3, a cyclotron accelerator may be used. In addition, in Embodiments 1 to 3, an example has been described in which the beam intensity is determined, and thereafter, it is determined whether to turn on or off the beam when the beam moves between the groups. However, the beam intensity may be determined after it is determined whether to turn on or off the beam when the beam moves between the groups.

### REFERENCE SIGNS LIST

1: SPOT DETERMINATION UNIT
2: GROUP CLASSIFICATION UNIT
3: PLANNING UNIT
5: PATIENT
10: TREATMENT PLANNING DEVICE
11: OVERALL CONTROL DEVICE
12: ACCELERATOR/BEAM TRANSPORT SYSTEM CONTROL DEVICE
13: IRRADIATION NOZZLE CONTROL DEVICE
20: ACCELERATOR
21: BEAM EMITTER
22: SYNCHROTRON ACCELERATOR
30: BEAM TRANSPORT SYSTEM
31: DEFLECTION ELECTROMAGNET
40: IRRADIATION NOZZLE
41A, 41B: SCANNING ELECTROMAGNET
42: IRRADIATION DOSE MONITOR
43: POSITION MONITOR
44: RIDGE FILTER
45: RANGE SHIFTER
50: TREATMENT TABLE
51: AFFECTED AREA
52: LAYER OF AFFECTED AREA TO BE IRRADIATED WITH SAME ENERGY
53: IRRADIATION SPOT
55: REMOTELY SEPARATED IRRADIATION SPOT
56-1, 2, 3: GROUP OF IRRADIATION SPOTS
61A, 61B: SCANNING ELECTROMAGNET POWER SUPPLY
71: SCANNING ELECTROMAGNET POWER SUPPLY CONTROL DEVICE
72: IRRADIATION DOSE MONITOR CONTROL DEVICE
73: POSITION MONITOR CONTROL DEVICE
81: BRAGG CURVE
82: SOBP (SPREAD OUT BRAGG PEAK)
90: CHARGED PARTICLE BEAM

1. A treatment planning device comprising:
a spot determination unit that classifies an irradiation region to be irradiated with a charged particle beam into a plurality of layers in an irradiation direction of the charged particle beam, and that arranges a plurality of irradiation spots which serve as irradiation positions of the charged particle beam, in the layers;
a group classification unit that classifies the irradiation spots into groups in accordance with at least either a distance between one irradiation spot and another irradiation spot which are arranged in the same layer or a target irradiation dose of each irradiation spot; and
a planning unit that prepares a plan so as to continuously emit the charged particle beam while the irradiation position is changed from an irradiation spot to a subsequent irradiation spot belonging to a certain group, and so as to stop emitting the charged particle beam while the irradiation position is changed from another irradiation spot belonging to another group to the irradiation spot belonging to the other group located in the same layer as that of the certain group.

2. The treatment planning device according to claim 1,
wherein the planning unit determines beam intensity for each of the groups classified by the group classification unit.

3. The treatment planning device according to claim 1,
wherein the group classification unit classifies an irradiation spot whose distance from a certain irradiation spot is equal to or greater than a threshold value, into another group.

4. The treatment planning device according to claim 1,
wherein for each group, the group classification unit determines a sequence of the irradiation spots to be irradiated with the charged particle beam.

5. The treatment planning device according to claim 1,
wherein the group classification unit determines a sequence of the irradiation spots to be irradiated with the charged particle beam, and classifies a certain irradiation spot into another group if a distance between the certain irradiation spot and the irradiation spot to be subsequently irradiated is equal to or greater than the threshold value.

6. The treatment planning device according to claim 1,
wherein the group classification unit classifies the irradiation spots whose target irradiation dose falls within a predetermined range, into the same group.

7. The treatment planning device according to claim 1,
wherein for each group, the planning unit determines whether to continuously emit the charged particle beam or to stop emitting the charged particle beam, while the irradiation position is changed from an irradiation spot to a subsequent irradiation spot belonging to a certain group.

8. A treatment planning method comprising:
a first step of classifying an irradiation region to be irradiated with a charged particle beam into a plurality of layers in an irradiation direction of the charged particle beam, and of performing spot arrangement for arranging a plurality of irradiation spots which serve as irradiation positions of the charged particle beam, in the layers;
a second step of classifying the irradiation spots into groups in accordance with at least either a distance between one irradiation spot and another irradiation spot which are arranged in the same layer or a target irradiation dose of each irradiation spot; and
a third step of outputting a signal to start to emit the charged particle beam while the irradiation position is changed from the irradiation spot to the subsequent irradiation spot belonging to a certain group, and of outputting a signal to stop emitting the charged particle beam while the irradiation position is changed from the irradiation spot belonging to a certain group to the irradiation spot belonging to another group located in the same layer as that of the certain group.

9. A control device that controls a charged particle beam to be emitted to a plurality of irradiation spots arranged in a plurality of layers into which an irradiation region to be irradiated with a charged particle beam is classified in an irradiation direction of the charged particle beam,
wherein the control device outputs a continuous beam emission signal so as to continuously emit the charged particle beam to the irradiation spots classified into groups in accordance with at least either a distance between one irradiation spot and another irradiation spot which are arranged in the same layer or a target irradiation dose of each irradiation spot, while an irradiation position is changed from an irradiation spot belonging to a certain group to a subsequent irradiation spot located in the same layer, and
wherein the control device outputs a beam stop signal so as to stop emitting the charged particle beam, while the irradiation position is changed from a irradiation spot belonging to a certain group to a irradiation spot belonging to another group located in the same layer as that of the certain group.

10. A particle beam treatment device comprising:
an acceleration device that accelerates a charged particle beam;
an irradiation device that emits the charged particle beam to a plurality of irradiation spots arranged in layers into which an irradiation region to be irradiated with the charged particle beam is classified in an irradiation direction of the charged particle beam; and
a control device that controls the acceleration device and the irradiation device,
wherein the control device outputs a continuous beam emission signal so as to continuously emit the charged particle beam to the irradiation spots classified into groups in accordance with at least either a distance between one irradiation spot and another irradiation spot which are arranged in the same layer or a target irradiation dose of each irradiation spot, while an irradiation position is changed from an irradiation spot belonging to a certain group to a subsequent irradiation spot, and
wherein the control device outputs a beam stop signal so as to stop emitting the charged particle beam, while the irradiation position is changed from an irradiation spot belonging to a certain group to an irradiation spot belonging to another group located in the same layer as that of the certain group.
